(19) United States
(12) Patent Application Publication
(10) Pub. No.: US 2025/0369837 A1
(43) Pub. Date: Dec. 4, 2025

(54) MODIFIED CORE SAMPLES FOR TESTING SCALE SQUEEZE

(71) Applicants: Chevron U.S.A. Inc., San Ramon, CA (US); The University of Tulsa, Tulsa, OK (US)

(72) Inventors: Wei Shi, Houston, TX (US); Mingdong Yuan, Missouri City, TX (US); Paul John Evans, Katy, TX (US); Jun Lu, Tulsa, OK (US)

(21) Appl. No.: 18/677,554

(22) Filed: May 29, 2024

(51) Int. Cl. G01N 1/28; G01N 33/24

(52) U.S. Cl. CPC G01N 1/286; G01N 33/24; G01N 2001/2873

(57) ABSTRACT

A method for preparing a subterranean core sample for testing scale squeeze can include obtaining a core sample, where the core sample comprises a proximal end, a distal end, and a fracture that continuously spans along its length between the proximal end and the distal end. The method can also include separating the core sample along the fracture into a first portion and a second portion. The method can further include placing a layer of proppant on a first fracture surface of the first portion that defines the fracture. The method can also include placing the second portion atop the first portion so that a second fracture surface of the second portion that defines the fracture contacts the layer of proppant. The method can further include enclosing the first portion, the second portion, and the layer of proppant in between with an enclosure along the length.

### TECHNICAL FIELD

The present application is related to core samples from subterranean formations and, more particularly, to modified core samples for testing scale squeeze.

### BACKGROUND

Scale squeeze is a practice sometimes used to prevent damage to a reservoir formation due to scale deposition at the near production wellbore area. Implementation of scale squeeze may rely on sufficient adsorption of a scale inhibitor on the formation rocks to enable slow release once production resumes after the treatment. Lab testing using formation cores is sometimes conducted prior to the field operations to evaluate scale inhibitor products and operational feasibility. Scale squeeze treatment into unconventional (e.g., shale, tight) formations, while conducted, is not well understood. Shale and other unconventional formations typically have a much lower permeability than conventional formations. As such, treatment of scale formations in unconventional plays is expected to only reach the fractures, which have much lower surface areas than conventional formation matrices. The adsorption of scale inhibitor on the fractures in scale formations is often not accurately represented by current lab testing methodologies using formation cores taken from unconventional (e.g., tight shale) subterranean formations and reservoirs.

### SUMMARY

In general, in one aspect, the disclosure relates to a method for preparing a subterranean core sample for testing scale squeeze. The method may include obtaining the subterranean core sample, where the core sample has a proximal end, a distal end, and a fracture that continuously spans along its length between the proximal end and the distal end. The method may also include separating the subterranean core sample along the fracture into a first portion and a second portion. The method may further include placing a layer of proppant on a first fracture surface of the first portion that defines the fracture. The method may also include placing the second portion atop the first portion so that a second fracture surface of the second portion that defines the fracture contacts the layer of proppant, and so that the proximal end of the first portion is adjacent to the proximal end of the second portion, and so that the distal end of the first portion is adjacent to the distal end of the second portion. The method may further include enclosing the first portion, the second portion, and the layer of proppant in between with an enclosure along the length, where the proximal end and the distal end are uncovered by the enclosure.

In another aspect, the disclosure relates to a modified core sample for testing scale squeeze. The modified core sample may include a first portion of a core sample having a first length defined by a first proximal end and a first distal end, where the first portion further comprises a first fracture surface disposed along the first length. The modified core sample may also include a second portion of a core sample having a second length defined by a second proximal end and a second distal end, where the second portion further includes a second fracture surface disposed along the second length, and where the second fracture surface complements the first fracture surface. The modified core sample may further include a layer of proppant placed between the first fracture surface and the second fracture surface. The modified core sample may also include a cover that encloses the first portion, the second portion, and the layer of proppant along the first length and the second length, where the first proximal end, the first distal end, the second proximal end, and the second distal end are uncovered by the cover.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, the same reference numerals used in different figures may designate like or corresponding but not necessarily identical elements.

FIGS. 1A through 1C show a field system, and details thereof, with which example embodiments may be used.

FIG. 2 shows the detail of FIG. 1C at a subsequent point in time according to certain example embodiments.

FIG. 3 shows the detail of FIG. 2 at a subsequent point in time according to certain example embodiments.

FIGS. 4A through 4C show various views of a core sample before being modified for testing scale squeeze according to certain example embodiments.

FIGS. 5 through 9 show side views of different core samples that have a fracture along their length before testing scale squeeze according to certain example embodiments.

FIGS. 10 through 14B show the process involved in modifying the core sample for testing scale squeeze according to certain example embodiments.

FIG. 15 shows a block diagram of a testing system 1519 that may perform testing of a modified core sample according to certain example embodiments.

FIG. 16 shows an example of a core sample assembly where the enclosure has been removed from the modified core sample 1500 of FIG. 15 according to certain example embodiments.

FIG. 17 shows a flowchart of a method for preparing a subterranean core sample for testing scale squeeze according to certain example embodiments.

### DETAILED DESCRIPTION

The example embodiments discussed herein are directed to systems, apparatus, methods, and devices for modified core samples for testing scale squeeze. Example embodiments may be used during certain types of field operations (e.g., fracturing, drilling, pre-production) in which core samples are obtained and regardless of which subterranean resource (e.g., oil, gas, water) is being produced. Example embodiments may be used with core samples from land-based or offshore operations. In addition, or in the alternative, example embodiments may be used with core samples taken from unconventional (e.g., tight shale) formations or conventional formations. While example embodiments may be directed to testing scale squeeze, modified core samples may additionally or alternatively be used for any of a number of other tests.

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of ±10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

A “subterranean formation” refers to practically any volume under a surface. For example, it may be practically any volume under a terrestrial surface (e.g., a land surface), practically any volume under a seafloor, etc. Each subsurface volume of interest may have a variety of characteristics, such as petrophysical rock properties, reservoir fluid properties, reservoir conditions, hydrocarbon properties, or any combination thereof. For example, each subsurface volume of interest may be associated with one or more of: temperature, porosity, salinity, permeability, water composition, mineralogy, hydrocarbon type, hydrocarbon quantity, reservoir location, pressure, etc. Those of ordinary skill in the art will appreciate that the characteristics are many, including, but not limited to, shale gas, shale oil, tight gas, tight oil, tight carbonate, carbonate, vuggy carbonate, unconventional (e.g., a permeability of less than 25 millidarcy (mD) such as a permeability of from 0.000001 mD to 25 mD)), diatomite, geothermal, mineral, etc. The terms “formation”, “subsurface formation”, “hydrocarbon-bearing formation”, “reservoir”, “subsurface reservoir”, “subsurface area of interest”, “subsurface region of interest”, “subsurface volume of interest”, and the like may be used synonymously. The term “subterranean formation” is not limited to any description or configuration described herein.

A “well” or a “wellbore” refers to a single hole, usually cylindrical, that is drilled into a subsurface volume of interest. A well or a wellbore may be drilled in one or more directions. For example, a well or a wellbore may include a vertical well, a horizontal well, a deviated well, and/or other type of well. A well or a wellbore may be drilled in the subterranean formation for exploration and/or recovery of resources. A plurality of wells (e.g., tens to hundreds of wells) or a plurality of wellbores are often used in a field depending on the desired outcome.

A well or a wellbore may be drilled into a subsurface volume of interest using practically any drilling technique and equipment known in the art, such as geosteering, directional drilling, etc. Drilling the well may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling tools, wireline tools, logging-while-drilling (LWD) tools, or other downhole tools. After drilling to a predetermined depth, the drill string and the drill bit may be removed, and then the casing, the tubing, and/or other equipment may be installed according to the design of the well. The equipment to be used in drilling the well may be dependent on the design of the well, the subterranean formation, the hydrocarbons, and/or other factors.

A well may include a plurality of components, such as, but not limited to, a casing, a liner, a tubing string, a sensor, a packer, a screen, a gravel pack, artificial lift equipment (e.g., an electric submersible pump (ESP)), and/or other components. If a well is drilled offshore, the well may include one or more of the previous components plus other offshore components, such as a riser. A well may also include equipment to control fluid flow into the well, control fluid flow out of the well, or any combination thereof. For example, a well may include a wellhead, a choke, a valve, and/or other control devices. These control devices may be located on the surface, in the subsurface (e.g., downhole in the well), or any combination thereof. In some embodiments, the same control devices may be used to control fluid flow into and out of the well. In some embodiments, different control devices may be used to control fluid flow into and out of a well. In some embodiments, the rate of flow of fluids through the well may depend on the fluid handling capacities of the surface facility that is in fluidic communication with the well. The equipment to be used in controlling fluid flow into and out of a well may be dependent on the well, the subsurface region, the surface facility, and/or other factors. Moreover, sand control equipment and/or sand monitoring equipment may also be installed (e.g., downhole and/or on the surface). A well may also include any completion hardware that is not discussed separately. The term “well” may be used synonymously with the terms “borehole,” “wellbore,” or “well bore.” The term “well” is not limited to any description or configuration described herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A.

In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C.

In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C).

In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of modified core samples for testing scale squeeze will be described more fully hereinafter with reference to the accompanying drawings, in which example embodiments of modified core samples for testing scale squeeze are shown. Modified core samples for testing scale squeeze may, however, be embodied in many different forms and should not be construed as limited to the example embodiments set forth herein. Rather, these example embodiments are provided so that this disclosure will be thorough and complete, and will fully convey the scope of modified core samples for testing scale squeeze to those of ordinary skill in the art. Like, but not necessarily the same, elements (also sometimes called components) in the various figures are denoted by like reference numerals for consistency.

Terms such as “first”, “second”, “primary,” “secondary,” “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation, and they are not meant to limit embodiments of modified core samples for testing scale squeeze. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

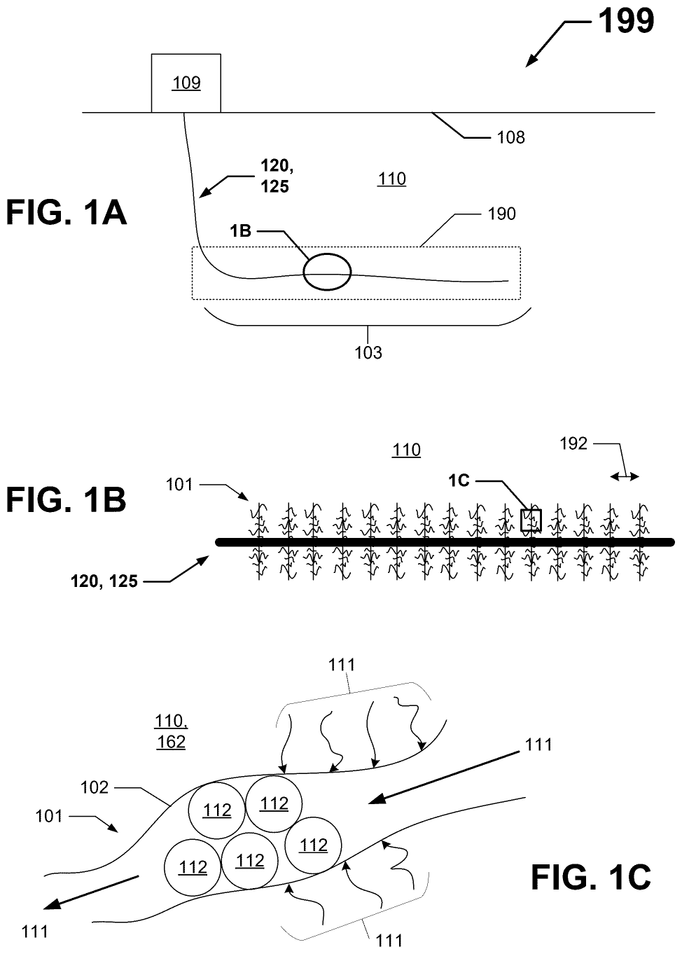

FIGS. 1A through 1C show a field system 199, including details thereof, from which core samples may be extracted and modified according to example embodiments for testing scale squeeze. Specifically, FIG. 1A shows a schematic diagram of a land-based field system 199 in which a wellbore 120 has been drilled in a subterranean formation 110. FIG. 1B shows a detail of a substantially horizontal section 103 of the wellbore 120 of FIG. 1A. FIG. 1C shows a detail of a created fracture 101 of FIG. 1B. The field system 199 in this example includes a wellbore 120 disposed in a subterranean formation 110 using field equipment 109 (e.g., a derrick, a tool pusher, a clamp, a tong, drill pipe, casing pipe, a drill bit, a wireline tool, a fluid pumping system) located above a surface 108 and within the wellbore 120. Once the wellbore 120 is drilled, a casing string 125 is inserted into the wellbore 120 to stabilize the wellbore 120 and allow for the extraction of subterranean resources (e.g., natural gas, oil) from the subterranean formation 110.

The surface 108 may be ground level for an onshore application and the sea floor/lakebed for an offshore application. For offshore applications, at least some of the field equipment may be located on a platform that sits above the water level. The point where the wellbore 120 begins at the surface 108 may be called the wellhead. While not shown in FIGS. 1A and 1B, there may be multiple wellbores 120, each with its own wellhead but that are located close to the other wellheads, drilled into the subterranean formation 110 and having substantially horizontal sections 103 that are close to each other. In such a case, the multiple wellbores 120 may be drilled at the same pad or at different pads. When the drilling process is complete, other operations, such as fracturing operations, may be performed. The fractures 101 are shown to be located in the horizontal section 103 of the wellbore 120 in FIG. 1B. The fractures 101, whether created and/or naturally occurring, may additionally or alternatively be located in other sections (e.g., a substantially vertical section, a transition area between a vertical section and a horizontal section) of the wellbore 120. Core samples that are modified according to certain example embodiments may be taken from any portion (e.g., the vertical portion, the horizontal portion 103) of the wellbore 120.

The subterranean formation 110 may include one or more of a number of formation types, including but not limited to shale, limestone, sandstone, clay, sand, and salt. In certain embodiments, a subterranean formation 110 may include one or more reservoirs in which one or more resources (e.g., oil, natural gas, water, steam) may be located. One or more of a number of field operations (e.g., fracturing, coring, tripping, drilling, setting casing, extracting downhole resources) may be performed to reach an objective of a user with respect to the subterranean formation 110.

The wellbore 120 may have one or more of a number of segments or hole sections, where each segment or hole section may have one or more of a number of dimensions. Examples of such dimensions may include, but are not limited to, a size (e.g., diameter) of the wellbore 120, a curvature of the wellbore 120, a total vertical depth of the wellbore 120, a measured depth of the wellbore 120, and a horizontal displacement of the wellbore 120. There may be multiple overlapping casing strings of various sizes (e.g., length, outer diameter) contained within and between these segments or hole sections to ensure the integrity of the wellbore construction. In this case, one or more of the segments of the subterranean wellbore 120 is the substantially horizontal section 103. As stated above, in additional or alternative cases, one or more of the segments of the subterranean wellbore 120 is a substantially vertical section.

As discussed above, inserted into and disposed within the wellbore 120 of FIGS. 1A and 1B are a number of casing pipes that are coupled to each other end-to-end to form the casing string 125. In this case, each end of a casing pipe has mating threads (a type of coupling feature) disposed thereon, allowing a casing pipe to be directly or indirectly mechanically coupled to another casing pipe in an end-to-end configuration. The casing pipes of the casing string 125 may be indirectly mechanically coupled to each other using a coupling device, such as a coupling sleeve.

Each casing pipe of the casing string 125 may have a length and a width (e.g., outer diameter). The length of a casing pipe may vary. For example, a common length of a casing pipe is approximately 40 feet. The length of a casing pipe may be longer (e.g., 60 feet) or shorter (e.g., 10 feet) than 40 feet. The width of a casing pipe may also vary and may depend on the cross-sectional shape of the casing pipe. For example, when the shape of the casing pipe is cylindrical, the width may refer to an outer diameter, an inner diameter, or some other form of measurement of the casing pipe. Examples of a width in terms of an outer diameter may include, but are not limited to, 4-½ inches, 7 inches, 7-⅝ inches, 8-⅝ inches, 10-¾ inches, 13-⅜ inches, and 14 inches.

The size (e.g., width, length) of the casing string 125 may be based on the information (e.g., diameter of the borehole drilled) gathered using field equipment with respect to the subterranean wellbore 120. The walls of the casing string 125 have an inner surface that forms a cavity that traverses the length of the casing string 125. Each casing pipe may be made of one or more of a number of suitable materials, including but not limited to steel. Cement is poured into the wellbore 120 through the cavity and then forced upward between the outer surface of the casing string 125 and the wall of the subterranean wellbore 120. In some cases, a liner may additionally be used with, or alternatively be used in place of, some or all of the casing pipes.

Once the cement dries to form concrete, a number of fractures 101 may be created in the subterranean formation 110. The fractures 101 may be created in any of a number of ways known in the industry, including but not limited to hydraulic fracturing, fracturing using electrodes, and/or other methods of creating fractures. The hydraulic fracturing process involves the injection of large quantities of fluids containing water, chemical additives, and proppants 112 into the subterranean formation 110 from the wellbore 120 to create fracture networks.

A subterranean formation 110 naturally has fractures 101, but these naturally occurring fractures 101 have inconsistent characteristics (e.g., length, spacing) and so in some cases may not be relied upon for extracting subterranean resources without having additional fractures 101, such as what is shown in FIG. 1B, created in the subterranean formation 110. Operations that create fractures 101 in the subterranean formation 110 use any of a number of fluids that include proppant 112 (e.g., sand, ceramic pellets). When proppant 112 is used, some of the fractures 101 (also sometimes called principal or primary fractures) receive proppant 112, while a remainder of the fractures 101 (also sometimes called secondary fractures) do not have any proppant 112 in them.

As shown in FIG. 1C, the proppant 112 is designed to become lodged inside at least some of the fractures 101 to keep those fractures 101 open after the fracturing operation is complete. The size of the proppant 112 is an important design consideration. Sizes (e.g., 40/70 mesh, 50/140 mesh) of the proppant 112 may vary. While the shape of the proppant 112 is shown as being uniformly spherical, and the size is substantially identical among the proppant 112, the actual sizes and shapes of the proppant 112 may vary. If the proppant 112 is too small, the proppant 112 will not be effective at keeping the fractures 101 open enough to effectively allow subterranean resources 111 to flow through the fractures 101 from the rock matrices 162 in the subterranean formation 110 to the wellbore 120. If the proppant 112 is too large, the proppant 112 may plug up the fractures 101, blocking the flow of the subterranean resources 111 through the fractures 101.

The use of proppant 112 in certain types of subterranean formation 110, such as shale, is important. Shale formations typically have permeabilities on the order of microdarcys (μD) to nanodarcys (nD). When fractures 101 are created in such formations with low permeabilities, it is important to sustain the fractures 101 and their conductivity for an extended period of time in order to extract more of the subterranean resource 111.

The various created fractures 101 that originate at the wellbore 120 and extend outward into the rock matrices 162 in the subterranean formation 110 in this case have consistent penetration lengths perpendicular to the wellbore 120 and have consistent coverage along at least a portion of the lateral length (substantially horizontal section) of the wellbore 120. For example, created fractures 101 may be 50 meters high and 200 meters long. Further, the created fractures 101 may be spaced a distance 192 apart from each other. The distance 192 (e.g., 25 meters, 5 meters, 12 meters) may be optimized based on the permeability and the porosity of the rock matrix 162 of the subterranean formation 110.

The created fractures 101 create a volume 190 within the subterranean formation 110 where the rock matrix 162 of the subterranean formation 110 is connected to the high conductivity fractures 101 located a short distance away. In addition to different configurations of the fractures 101, other factors that may contribute to the viability of the subterranean formation 110 may include, but are not limited to, permeability of the rock matrix 162, capillary pressure, and the temperature and pressure of the subterranean formation 110. Each fracture 101, whether created or naturally occurring, is defined by a boundary known as a frac face 102. The frac face 102 provides a transition between the paths formed by the rock matrices 162 in the subterranean formation 110 and the fracture 101. The subterranean resources 111 flow through the paths formed by the rock matrices 162 in the subterranean formation 110 into the fracture 101.

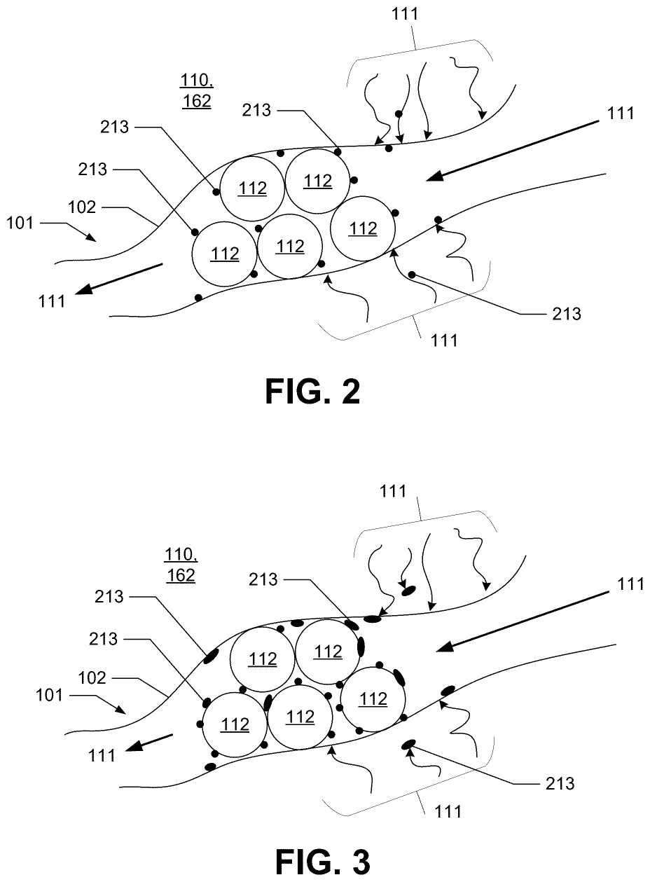

FIG. 2 shows the detail of FIG. 1C at a subsequent point in time relative to what is captured in FIG. 1C. FIG. 3 shows the detail of FIG. 2 at a subsequent point in time relative to what is captured in FIG. 2. For example, FIG. 2 may show the detail of FIG. 1C six months later than the time captured in FIG. 1C after flowing a scale enhancer (a type of fluid) therethrough, and FIG. 3 may show the detail of FIG. 2 four year later than the time captured in FIG. 2 after continuing to flow the scale enhancer therethrough. Referring to FIGS. 1A through 3, the detail in FIG. 2 shows, in addition to the proppant 112 within the fracture 101, a subterranean resource 111 (e.g., natural gas, oil) is shown flowing within the fracture 101 from the rock matrix 162, around the proppant 112 in the fracture 101, and on to the wellbore 120.

As the subterranean resource 111 flows within the paths formed by the rock matrices 162 and around or on the proppant 112 in the fracture 101, scale deposition 213 may occur (e.g., scale particles formed during the shut-in stage before the well is put on production) on the pore throat within the rock matrices 162, on the proppant 112, and/or on the frac face 102. (It should be noted that while FIGS. 2 and 3 refer to scale deposition 213, element 213 described herein may more generally refer to any type of solid, which may also include, but is not limited to, asphaltenes, sludge, and fines). Over time, the scale depositions 213 may begin to accumulate on the rock matrices 162, on the proppant 112, and/or on the frac face 102. In some cases, at least some of the scale depositions 213 may be an inorganic deposit from ionic materials in water that attaches to solid surfaces. Hydrocarbons may be adsorbed on scale depositions 213. Under field conditions, scale depositions 213 may be a mixture of inorganic and organic components.

Scale depositions 213 may be initiated during a prior phase (e.g., completion) of a field operation, where fluids and chemicals used downhole may interact with formation rock (e.g., the frac face 102, the rock matrices 162), resulting in the mobilization and release of elements from the rock matrices 162 adjacent to the fractures 101, and comingle with formation water in and/or near perforations and along fractures 101. Later, in a subsequent phase (e.g., shutting in) of the field operation, the rock-fluid interaction and the commingling of different fluids may lead to the formation (crystallization) and growth of scale depositions 213 in or near the perforations, the rock matrices 162, and the fractures 101. In yet another subsequent phase (e.g., production) of the field operation, the degradation in the conductivity and production flow path integrity over time in the rock matrices 162 and the fractures 101, caused by agglomerate build up of scale depositions 213, may lead to plugging in or near the perforations, rock matrices 162, fractures 101, and completion tools.

The scale depositions 213 that accumulate within the rock matrices 162 and the fractures 101 may be composed of one or more of any of a number of compounds, including but not limited to calcium carbonate, barium sulfate, calcium sulfate, strontium sulfate, iron carbonate, iron oxide, iron sulfide, other oxides, other sulfides, other carbonates, other sulfates, halides, and hydroxides. While the scale depositions 213 may additionally or alternatively be composed of other compounds (e.g., gas hydrates, organic deposits (e.g., asphaltenes, waxes, acid induced sludges), and naphthenates), testing of the modified core samples according to example embodiments may, in some cases, focus on the reduction of scale depositions 213 caused by inorganic deposits. The scale depositions 213 may be caused by one or more of any of a number of factors, including but not limited to supersaturation, mixing incompatible ions, changes in temperature, changes in pressure, carbon dioxide interaction, and a change in the pH of water in the fluid.

Scale depositions 213 may form during the shut-in stage prior to the well being put into production, as shown in FIG. 2. In such a case, the scale depositions 213 deposited on the rock matrices 162, on the proppant 112, and on the frac face 102 may be small and spotty. As a result, the scale depositions 213 may not contribute much to inhibiting the flow of the subterranean resource 111 through the paths within the rock matrices 162 and around the proppant 112 within the fracture 101 formed by the frac face 102. In the portion of the fracture 101 shown at the time captured in FIG. 2, there are 2 separate scale depositions 213 within the rock matrices 162, 8 scale depositions 213 on the proppant 112, and 4 scale depositions 213 on the frac face 102. The number, size, and location of the scale depositions 213 within the rock matrices 162 and the fracture 101 may vary.

When the well is put on production, some scale depositions 213 may stay at their original position, while some scale particles may move/migrate together with the produced water and deposit at another location along the production pathway. As more water is produced, if no mitigation efforts are made, the existing scale depositions 213 may increase in size and new scale depositions 213 may develop over time. An example of this is captured in FIG. 3, which shows that the scale depositions 213 become larger and less spotty. As a result, the scale depositions 213 in FIG. 3 begin to contribute to inhibiting the flow of the subterranean resource 111 (e.g., a hydrocarbon) along the paths formed by the rock matrices 162, through the frac face 102 (impacting migration of the subterranean resource 111 from the rock matrix 162), and around the proppant 112 (combined with the scale depositions 213 on the proppant 112 and on the frac face 102) within the fracture 101.

In the portion of the fracture 101 shown at the time captured in FIG. 3, there are 25 separate scale depositions 213 within the rock matrices 162, at the frac face 102, and on the proppant 112, many of which are significantly larger than the size of the scale depositions 213 shown in FIG. 2. Also, some of the scale depositions 213 in FIG. 3 have migrated to a new location relative to their location in FIG. 2. Again, the number, size, and location of the scale depositions 213 within the fracture 101 may vary. Modifying core samples according to example embodiments and testing those modified core samples for testing scale squeeze may be designed in some cases to analyze the type of inorganic material in the scale depositions 213 in a particular experiment or field condition of a field operation. Modifying core samples according to example embodiments and testing those modified core samples for testing scale squeeze also designed to determine the optimal way to reduce (e.g., remediate (e.g., removal of scale depositions 213 with a chemical treatment in the form of a fluid (e.g., an acid, a chelant)), mitigate) the development and accumulation of the scale depositions 213 in that particular field operation.

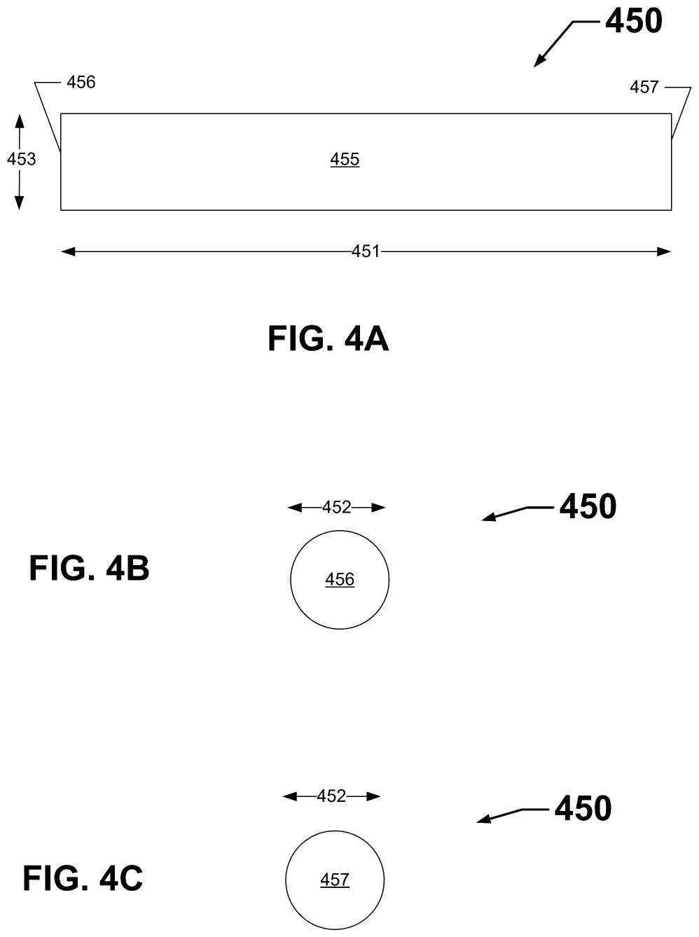

FIGS. 4A through 4C show various views of a core sample 450 before being modified for testing scale squeeze according to certain example embodiments. Specifically, FIG. 4A shows a side view of the core sample 450. FIG. 4B shows a front view of the core sample 450. FIG. 4C shows a rear view of the core sample 450. Referring to the description above with respect to FIGS. 1A through 3, the core sample 450 of FIGS. 4A through 4C has a body 455 with a proximal end 456 and a distal end 457. The body 455 of the core sample 450 in this case is a cylinder having a height 453, a width 452, and a length 451 bounded by the proximal end 456 and the distal end 457. Because of the cylindrical shape of the body 455, the height 453 and the width 452 are substantially the same as each other in this case. In this example, the proximal end 456 of the body 455 is substantially perpendicular to the outer perimeter of the body, and the distal end 457 of the body 455 is substantially parallel to the proximal end 456.

In alternative embodiments, the body 455 of the core sample 450 can have any of a number of other shapes (e.g., an elongated three-dimensional rectangle, a cube, an elongated hexagon). In addition, or in the alternative, while the width 452 and the height 453 of the core sample is substantially uniform along the length 451 of the body 455 of the core sample 450 in this case, in alternative embodiments the width 452 and/or the height 453 of the core sample 450 may vary along some or all of the length 451 of the body 455. In addition, or in the alternative, the cross sectional shape (in this case, a circle) of the body 455 of the core sample 450 may vary along some or all of the length 451 of the body 455 of the core sample 450.

A core sample (e.g., core sample 450) is taken from a formation layer (e.g., shale) of a subterranean formation (e.g., subterranean formation 110) using a tool (e.g., a coring tool) in a wellbore (e.g., wellbore 120). Once a core sample (e.g., core sample 450) is obtained (e.g., retrieved directly from the wellbore (e.g., wellbore 120), received from an entity that retrieves the core sample directly from the wellbore), the core sample is separated, according to example embodiments, into multiple portions along a lengthwise fracture in the body (e.g., body 455) of the core sample. Such a fracture along the length of the body of the core sample may be naturally occurring (e.g., existing at the time the core sample is extracted) and/or created (e.g., cut, stress induced) after the core sample is extracted.

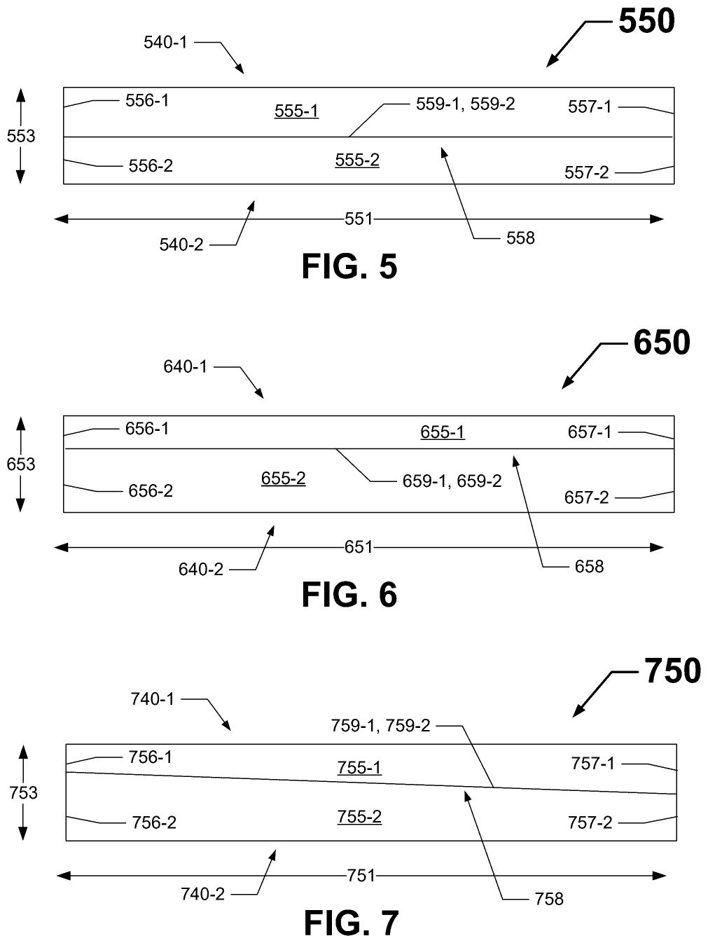

FIGS. 5 through 9 show side views of different core samples that have a fracture along their length before testing scale squeeze according to certain example embodiments. Referring to the description above with respect to FIGS. 1A through 4C, the core sample 550 of FIG. 5 may be substantially similar to the core sample 450 of FIGS. 4A through 4C. For example, the core sample 550 of FIG. 5 has a body 555 with an overall length 551 and an overall height 553. In this case, the body 555 is cylindrical in form, so the overall height 553 is substantially the same as the overall width (e.g., similar to the width 452 of the body 455 of the core sample 450 discussed above). The proximal end 556 of the body 555 is substantially perpendicular to the outer perimeter of the body 555, and the distal end 557 of the body 555 is substantially parallel to the proximal end 556.

In this case, the core sample 550 has two portions 540 (portion 540-1 and portion 540- 2). Portion 540-1 and portion 540-2 are separated by a fracture 558, which in this case is created by cutting (e.g., using a saw) the body 555 along its length 551. In this particular example, the fracture 558 is planar and runs from the proximal end 556 to the distal end 557 of the body 555. Also, in this case, the fracture 558 is substantially parallel to the outer perimeter of the body 555. Further, the fracture 558 runs substantially halfway of the height 553 of the body 555, which means that the shape and size of portion 540-1 and the shape and size of portion 540-2 are substantially the same as each other. The fracture 558 generates a fracture surface 559-1 that bounds the bottom of portion 540-1 between the proximal end 556-1 and the distal end 557-1 of the body 555-1 of the portion 540-1. Similarly, the fracture 558 generates a fracture surface 559-2 that bounds the top of portion 540-2 between the proximal end 556-2 and the distal end 557-2 of the body 555-2 of the portion 540-2.

The core sample 650 of FIG. 6 may be substantially similar to the core sample 450 of FIGS. 4A through 4C. For example, the core sample 650 of FIG. 6 has a body 655 with an overall length 651 and an overall height 653. In this case, the body 655 is cylindrical in form, so the overall height 653 is substantially the same as the overall width (e.g., similar to the width 452 of the body 455 of the core sample 450 discussed above). The proximal end 656 of the body 655 is substantially perpendicular to the outer perimeter of the body 655, and the distal end 657 of the body 655 is substantially parallel to the proximal end 656.

In this case, the core sample 650 has two portions 640 (portion 640-1 and portion 640-2). Portion 640-1 and portion 640-2 are separated by a fracture 658, which in this case is created by cutting (e.g., using a saw) the body 655 along its length 651. In this particular example, the fracture 658 is planar and runs from the proximal end 656 to the distal end 657 of the body 655. Also, in this case, the fracture 658 is substantially parallel to the outer perimeter of the body 655. Further, the fracture 658 runs substantially two-third of the height 653 of the body 655, which means that the shape and size of portion 640-1 is different than and smaller than the shape and size of portion 640-2. The fracture 658 generates a fracture surface 659-1 that bounds the bottom of portion 640-1 between the proximal end 656-1 and the distal end 657-1 of the body 655-1 of the portion 640-1. Similarly, the fracture 658 generates a fracture surface 659-2 that bounds the top of portion 640-2 between the proximal end 656-2 and the distal end 657-2 of the body 655-2 of the portion 640-2.

The core sample 750 of FIG. 7 may be substantially similar to the core sample 450 of FIGS. 4A through 4C. For example, the core sample 750 of FIG. 7 has a body 755 with an overall length 751 and an overall height 753. In this case, the body 755 is cylindrical in form, so the overall height 753 is substantially the same as the overall width (e.g., similar to the width 452 of the body 455 of the core sample 450 discussed above). The proximal end 756 of the body 755 is substantially perpendicular to the outer perimeter of the body 755, and the distal end 757 of the body 755 is substantially parallel to the proximal end 756.

In this case, the core sample 750 has two portions 740 (portion 740-1 and portion 740- 2). Portion 740-1 and portion 740-2 are separated by a fracture 758, which in this case is created by cutting (e.g., using a saw) the body 755 along its length 751. In this particular example, the fracture 758 is planar and runs from the proximal end 756 to the distal end 757 of the body 755. Also, in this case, the fracture 758 is antiparallel to the outer perimeter of the body 755. Further, the fracture 758 runs from substantially two-thirds of the height 753 of the proximal end 756 of the body 755 to substantially half the height 753 of the distal end 757 of the body 755. As a result, the shape and size of portion 740-1 is different than and smaller than, respectively, the shape and size of portion 740-2. The fracture 758 generates a fracture surface 759-1 that bounds the bottom of portion 740-1 between the proximal end 756-1 and the distal end 757-1 of the body 755-1 of the portion 740-1. Similarly, the fracture 758 generates a fracture surface 759-2 that bounds the top of portion 740-2 between the proximal end 756-2 and the distal end 757-2 of the body 755-2 of the portion 740-2.

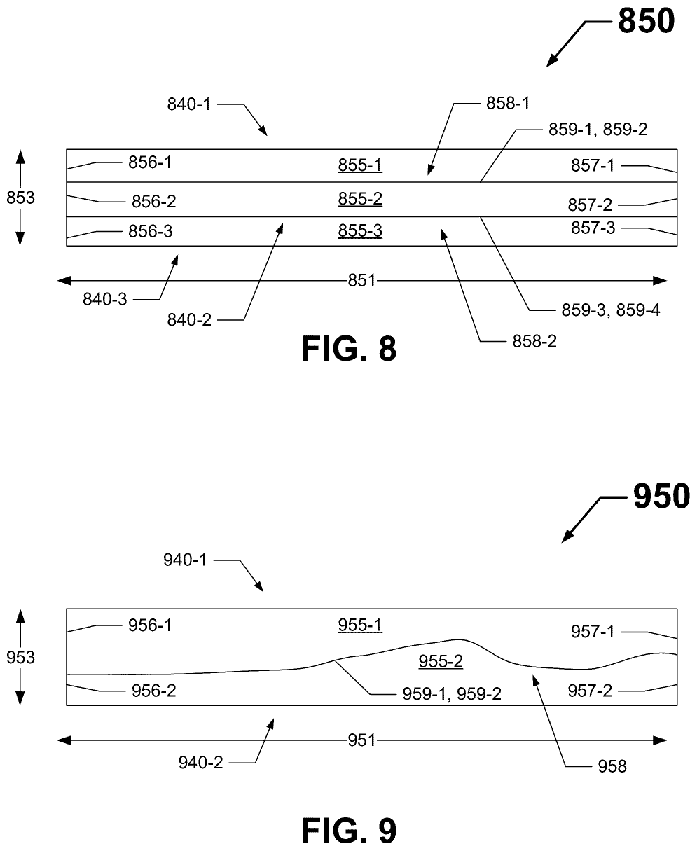

The core sample 850 of FIG. 8 may be substantially similar to the core sample 450 of FIGS. 4A through 4C. For example, the core sample 850 of FIG. 8 has a body 855 with an overall length 851 and an overall height 853. In this case, the body 855 is cylindrical in form, so the overall height 853 is substantially the same as the overall width (e.g., similar to the width 452 of the body 455 of the core sample 450 discussed above). The proximal end 856 of the body 855 is substantially perpendicular to the outer perimeter of the body 855, and the distal end 857 of the body 855 is substantially parallel to the proximal end 856.

In this case, the core sample 850 has three portions 840 (portion 840-1, portion 840-2, and portion 840-3). Portion 840-1 and portion 840-2 are separated by a fracture 858-1, and portion 840-2 and portion 840-3 are separated by a fracture 858-2. In this case, fracture 858-1 and fracture 858-2 are created by cutting (e.g., using a saw) the body 855 along its length 851. In this particular example, the fracture 858-1 and the fracture 858-2 are planar and run from the proximal end 856 to the distal end 857 of the body 855. Also, in this case, the fracture 858-1 and the fracture 858-2 are substantially parallel to the outer perimeter of the body 855. Further, the fracture 858-1 runs substantially two-thirds of the height 853 of the body 855, and the fracture 858-2 runs substantially one-third of the height 853 of the body 855. As a result, the shape and size of portion 840-1 and portion 840-3 are substantially the same as each other and different than the shape and size of portion 840-2.

The fracture 858-1 generates a fracture surface 859-1 that bounds the bottom of portion 840-1 between the proximal end 856-1 and the distal end 857-1 of the body 855-1 of the portion 840-1. Similarly, the fracture 858 generates a fracture surface 859-2 that bounds the top of portion 840-2 between the proximal end 856-2 and the distal end 857-2 of the body 855-2 of the portion 840-2. The fracture 858-2 generates a fracture surface 859-2 that bounds the bottom of portion 840-2 between the proximal end 856-2 and the distal end 857-2 of the body 855-2 of the portion 840-2. Similarly, the fracture 858-2 generates a fracture surface 859-3 that bounds the top of portion 840-3 between the proximal end 856-3 and the distal end 857-3 of the body 855-3 of the portion 840-3.

The core sample 950 of FIG. 9 may be substantially similar to the core sample 450 of FIGS. 4A through 4C. For example, the core sample 950 of FIG. 9 has a body 955 with an overall length 951 and an overall height 953. In this case, the body 955 is cylindrical in form, so the overall height 953 is substantially the same as the overall width (e.g., similar to the width 452 of the body 455 of the core sample 450 discussed above). The proximal end 956 of the body 955 is substantially perpendicular to the outer perimeter of the body 955, and the distal end 957 of the body 955 is substantially parallel to the proximal end 956.

In this case, the core sample 950 has two portions 940 (portion 940-1 and portion 940-2). Portion 940-1 and portion 940-2 are separated by a fracture 958, which in this case is naturally occurring and spans the length 951 of the body 955. In this particular example, the fracture 958 is random and runs from the proximal end 956 to the distal end 957 of the body 955. The fracture 958 runs from substantially one-third of the height 953 of the proximal end 956 of the body 955 to substantially half the height 953 of the distal end 957 of the body 955. As a result, the shape and size of portion 940-1 is different than the shape and size of portion 940-2. The fracture 958 generates a fracture surface 959-1 that bounds the bottom of portion 940-1 between the proximal end 956-1 and the distal end 957-1 of the body 955-1 of the portion 940-1. Similarly, the fracture 958 generates a fracture surface 959-2 that bounds the top of portion 940-2 between the proximal end 956-2 and the distal end 957-2 of the body 955-2 of the portion 940-2.

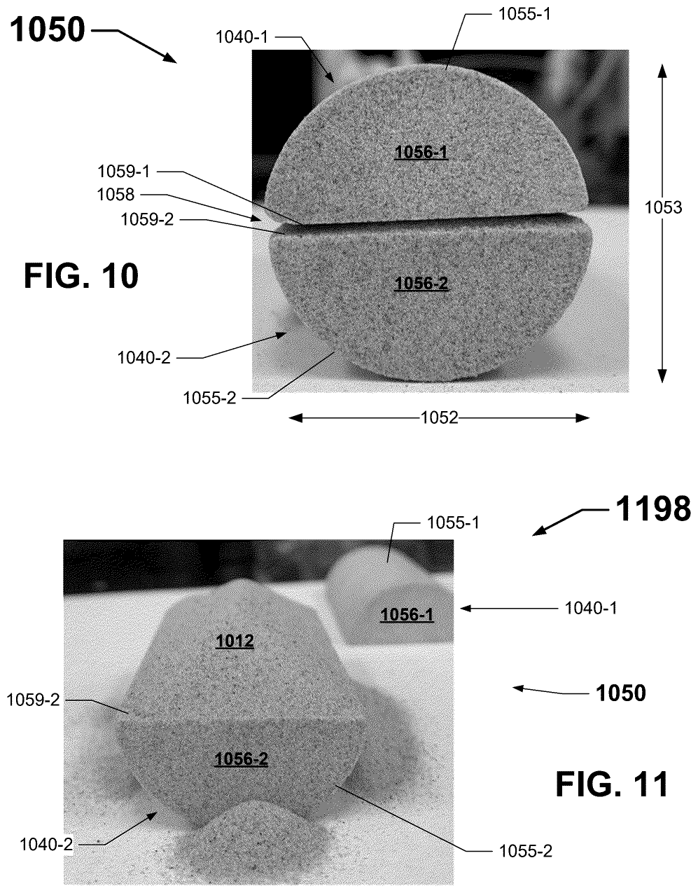

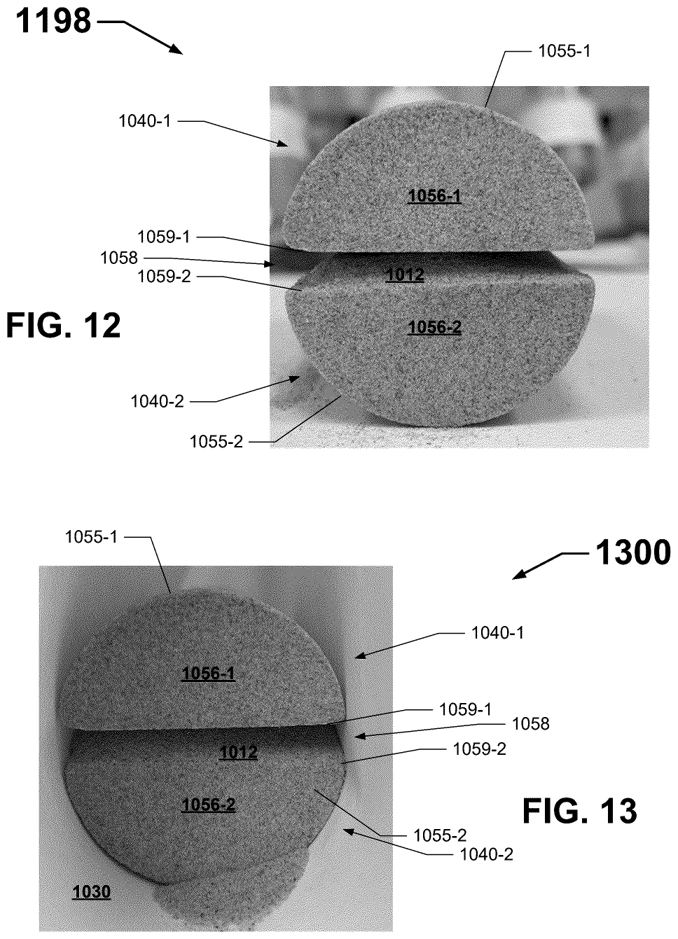

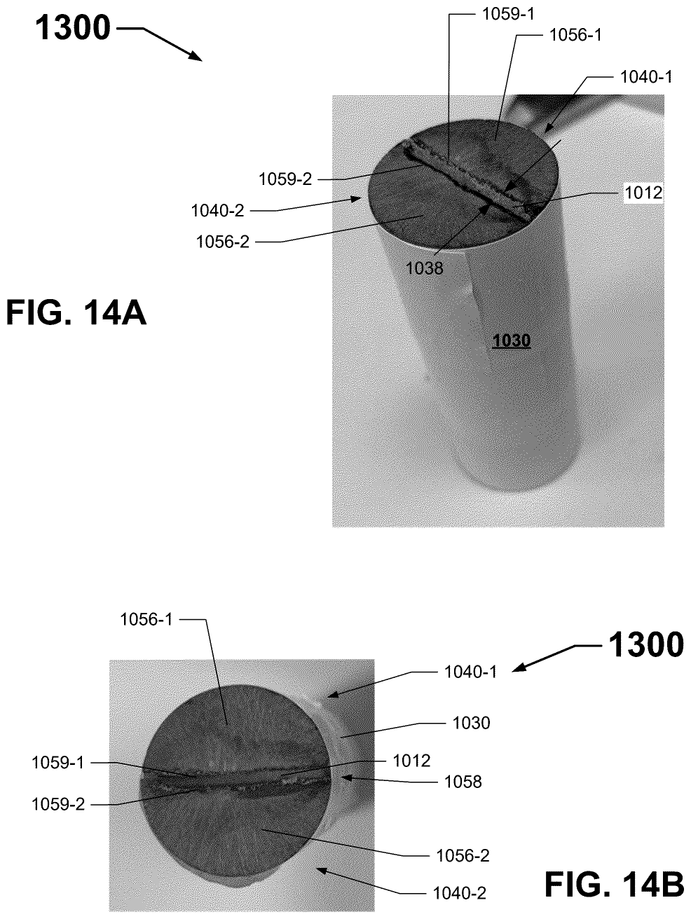

According to example embodiments, when a core sample (e.g., core sample 550) has multiple portions (e.g., portions 540), the core sample is modified for testing scale squeeze. FIGS. 10 through 14B show the process involved in modifying (also sometimes called preparing) the core sample for testing scale squeeze according to certain example embodiments. Specifically, FIG. 10 shows a front view of a core sample 1050. FIG. 11 shows a front view of a core sample assembly 1198. FIG. 12 shows a front view of the core sample assembly 1198 at a time subsequent to the time captured in FIG. 11. FIG. 13 shows a modified core sample 1300 in preassembly. FIGS. 14A and 14B show a front-side view and a front view, respectively, of the modified core sample 1300.

Referring to the description of FIGS. 1A through 9 above, FIG. 10 shows an image of an actual core sample 1050 that is configured substantially similar to the core sample 550 of FIG. 5 above. For example, the core sample 1050 of FIG. 10 includes a body 1055 with an overall length (similar to the length 551 of the core sample 550), an overall width 1052, and an overall height 1053. In this case, the body 1055 is cylindrical in form, so the overall height 1053 is substantially the same as the overall width 1052. The proximal end 1056 of the body 1055 is substantially perpendicular to the outer perimeter of the body 1055, and the distal end (substantially similar to the distal end 557 of the core sample 550) of the body 1055 is substantially parallel to the proximal end 1056.

In this case, the core sample 1050 has two portions 1040 (portion 1040-1 and portion 1040-2). Portion 1040-1 and portion 1040-2 are separated by a fracture 1058, which in this case is created by cutting (e.g., using a saw) the body 1055 along its length. In this particular example, the fracture 1058 is planar and runs from the proximal end 1056 to the distal end of the body 1055. Also, in this case, the fracture 1058 is substantially parallel to the outer perimeter of the body 1055. Further, the fracture 1058 runs substantially halfway of the height 1053 of the body 1055, which means that the shape and size of portion 1040-1 and the shape and size of portion 1040-2 are substantially the same as each other. The fracture 1058 generates a fracture surface 1059-1 that bounds the bottom of portion 1040-1 between the proximal end 1056-1 and the distal end of the body 1055-1 of the portion 1040-1. Similarly, the fracture 1058 generates a fracture surface 1059-2 that bounds the top of portion 1040-2 between the proximal end 1056-2 and the distal end of the body 1055-2 of the portion 1040-2.

In FIG. 11, which captures a time subsequent to the time captured in FIG. 10, portion 1040-1 is separated from portion 1040-2 and set aside, leaving the fracture surface 1059-2 of portion 1040-2 exposed. A user (e.g., an engineer, a lab technician, a consultant, a company representative) then places (e.g., pours) proppant 1012 over the entire fracture surface 1059-2 of portion 1040-2. In alternative embodiments, portion 1040-2 is separated from portion 1040-1 and set aside, leaving the fracture surface 1059-1 of portion 1040-1 exposed. A user (e.g., an engineer, a lab technician, a consultant, a company representative) may then place (e.g., pour) proppant 1012 over the entire fracture surface 1059-1 of portion 1040-1. The proppant 1012 may be substantially the same as the proppant 112 discussed above.

In FIG. 12, which captures a time subsequent to the time captured in FIG. 11, portion 1040-1 is placed on top of (e.g., replaced) portion 1040-2 so that the fracture surface 1059-1 of the portion 1040-1 that defines the fracture 1058 contacts the proppant 1012 and forms a layer of proppant 1012 along substantially all of the fracture 1058. In alternative embodiments, portion 1040-2 is placed on top of (e.g., replaced) portion 1040-1 so that the fracture surface 1059-2 of the portion 1040-2 that defines the fracture 1058 contacts the proppant 1012 and forms a layer of proppant 1012 along substantially all of the fracture 1058.

In either case, when portion 1040-1 is placed on top of portion 1040-2 (or vice versa), the orientation may be such that the proximal end 1056-1 of portion 1040-1 is adjacent to the proximal end 1056-2 of portion 1040-2, and so that the distal end of portion 1040-1 is adjacent to the distal end of portion 1040-2, as shown in FIG. 12. Alternatively, when portion 1040-1 is placed on top of portion 1040-2 (or vice versa), the orientation may be such that the proximal end 1056-1 of portion 1040-1 is adjacent to the distal end of portion 1040-2, and so that the distal end of portion 1040-1 is adjacent to the proximal end 1056-2 of portion 1040-1. As portion 1040-1 and portion 1040-2 are compressed toward each other, the layer of proppant 1012 becomes compacted and uniform, and excess proppant 1012 spills out of the space defined by the fracture surface 1059-1 of portion 1040-1 and the fracture surface 1059-2 of portion 1040-2.

In FIG. 13, the core sample assembly 1198 is placed on an enclosure 1030. The enclosure 1030 is configured to retain the components (specifically, the layer of proppant 1012, portion 1040-1, and portion 1040-2) of the core sample assembly 1198 in a secure position relative to each other. The enclosure 1030 may be or include any of a number of materials, including but not limited to metal, plastic, a polymer, rubber, and ceramic. The enclosure 1030 may be rigid, flexible, malleable, permeable, impermeable, heat activated (e.g., shrink wrap), and/or have any other characteristics needed to maintain the modified core sample (shown in FIGS. 14A and 14B below) throughout scale squeeze testing. The enclosure 1030 may be a single piece or multiple pieces.

In FIGS. 14A and 14B, the modified core sample 1300 of FIG. 13 is completed when manipulation of the enclosure 1030 is complete. In this case, the enclosure 1030 encloses portion 1040-1, portion 1040-2, and the layer of proppant 1012 in between. The enclosure 1030 wraps around the outer perimeter of the portions 1040 along their length, which leaves the proximal end 1056 (specifically, proximal end 1056-1 for portion 1040-1 and proximal end 1056-2 for portion 1040-2) and the distal end uncovered by the enclosure 1030. The layer of proppant 1012 has a thickness 1038 that may be substantially the same along the length of the portions 1040.

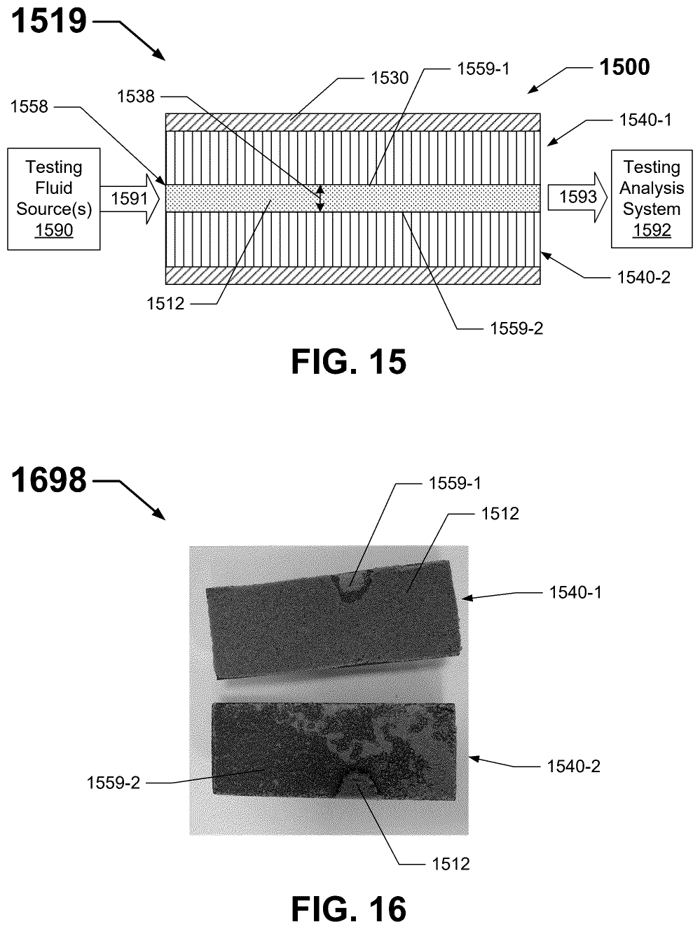

When the modified core sample (e.g., modified core sample 1300 of FIGS. 14A and 14B) is completely assembled, testing (e.g., scale squeeze testing) may be performed on the modified core sample. FIG. 15 shows a block diagram of a testing system 1519 that may perform testing of a modified core sample 1500 according to certain example embodiments. Referring to the description above with respect to FIGS. 1A through 14B, the testing system 1519 includes a modified core sample 1500, a testing fluid source 1590, a testing fluid 1591, an exit fluid 1593, and a testing analysis system 1592.

The modified core sample 1500 of FIG. 15 is substantially similar to the modified core sample (including components thereof) discussed above. Specifically, the modified core sample 1500 includes portion 1540-1 and portion 1540-2 that have substantially the same shape and size as each other. A fracture 1558 in the form of a cut is filled with a layer of proppant 1512, which is abutted by the fracture surface 1559-1 of portion 1540-1 and the fracture surface 1559-2 of portion 1540-2. The layer of proppant 1512 has a thickness 1538 that may be substantially the same along the length of the portions 1540. The layer of proppant 1512, portion 1540-1, and portion 1540-2 are enclosed along their length by an enclosure 1530.

The one or more testing fluid sources 1590 of the testing system 1519 may be or include any natural (e.g., a lake or other body of water, ambient air) and/or man-made (e.g., a tank, a container) medium in which some (e.g., a component, an ingredient, a compound) or all of the testing fluid 1591 may be found and/or contained. The one or more testing fluid sources 1590 may also include one or more additional components (e.g., a pump, a motor, a compressor, piping, a valve, a gauge, a controller, a protective relay, a power source) to deliver the testing fluid 1591 to the proximal end of the modified core sample 1500.

The testing fluid 1591 may be or include a liquid and/or a gas. In some cases, the testing fluid 1591 may also include one or more solids. For example, the testing fluid 1591 may be a reagent alcohol that includes a mixture of ethyl alcohol, isopropyl alcohol, methanol, and water. When the testing fluid 1591 reaches the modified core sample 1500, it travels through the layer of proppant 1512 between the fracture surface 1559-1 of portion 1540-1 and the fracture surface 1559-2 of portion 1540-2 at the proximal end. As the testing fluid 1591 passes through the layer of proppant 151280030, it transforms into the exit fluid 1593 as it leaves the distal end of the modified core sample 1500.

The exit fluid 1593 may then be tested by the testing analysis system 1592, which may include any equipment (e.g., meters, valves, piping, sensor devices, chemical additives, filters, pumps, motors, controllers, centrifuges, agitators) that may be used to test and analyze the exit fluid 1593. In addition, or it the alternative, some or all of the testing analysis system 1592 may be used to test and analyze the layer of proppant (e.g., layer of proppant 1512), one or more of the portions (e.g., portions 1540), the enclosure (e.g., enclosure 1530) and/or any other part of the modified core sample (e.g., modified core sample 1500).

As discussed above, the modified core sample (e.g., modified core sample 1500) may be tested and analyzed after test fluid (e.g., test fluid 1591) has flowed therethrough. FIG. 16 shows an example of a core sample assembly 1698 where the enclosure 1530 has been removed from the modified core sample 1500 of FIG. 15 according to certain example embodiments. After the enclosure 1530 is removed, portion 1540-1 and portion 1540-2 are separated from each other. Due to compaction and the test fluid 1591 that has flowed during testing, the layer of proppant 1512 remains largely intact when the portions 1540 of the core sample assembly 1698 are separated from each other. The layer of proppant 1512 may then be tested and analyzed using the testing analysis system 1592.

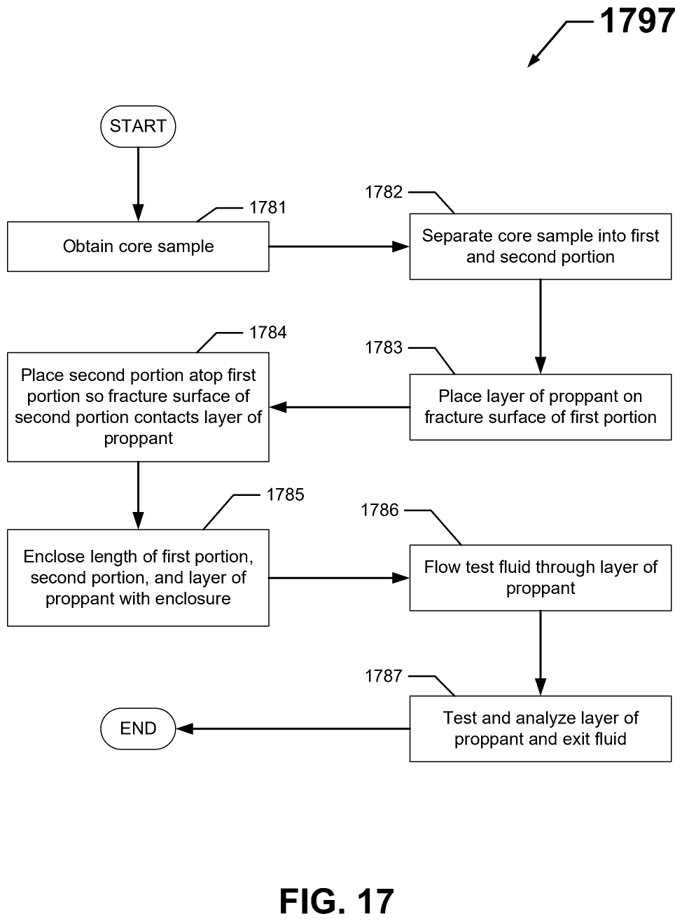

FIG. 17 shows a flowchart 1797 of a method for preparing a subterranean core sample for testing scale squeeze according to certain example embodiments. While the various steps in this flowchart 1797 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order. Some or all of the steps of the method of FIG. 17 may be performed off site (e.g., in a laboratory remote from a subterranean formation being evaluated). In addition, or in the alternative, some or all of the steps of the method of FIG. 17 may be performed on site where a subterranean formation is being evaluated.

In addition, a person of ordinary skill in the art will appreciate that additional steps not shown in FIG. 17 may be included in performing this method. Accordingly, the specific arrangement of steps should not be construed as limiting the scope of this disclosure. Any of the functions in the method may be performed by a user (e.g., a lab technician, a consultant, an engineer). The method shown in FIG. 17 is merely an example that may be performed by using an example system described herein. In other words, systems preparing a subterranean core sample for testing scale squeeze may perform other functions using other methods in addition to and/or aside from those shown in FIG. 17. Referring to the description above with respect to FIGS. 1A through 16, the method shown in the flowchart 1797 of FIG. 17 begins at the START step and proceeds to step 1781, where a core sample 450 is obtained. The core sample 450 may be obtained directly by the user performing one or more of the remaining steps in the flowchart 1797. Alternatively, the core sample 450 may be obtained indirectly through another user or other entity involved in procuring the core sample 450. An example of this step 1781 and associated details are provided above with respect to FIGS. 4A through 4C and associated description.

In step 1782, the core sample 550 is separated into at least two portions 540. The portions 540 may be bounded by fracture surfaces 559 caused by a fracture 558, where the fracture 558 may be created (e.g., cutting with a saw) or naturally occurring. The fracture 558 may be continuous from the proximal end 556 to the distal end 557 of the core sample 550. Examples of this step 1782 and associated details are provided above with respect to FIGS. 5 through 10 and associated description. In step 1783, a layer of proppant 1012 is placed on a fracture surface 1059 of one of the portions 1040. An example of this step 1783 and associated details are provided above with respect to FIG. 11 and associated description.

In step 1784, the other portion 1040 is placed atop the layer of proppant 1012 so that the fracture surface 1059 contacts the layer of proppant 1012. An example of this step 1784 and associated details are provided above with respect to FIG. 12 and associated description. In step 1785, the length of the portions 1040 and the layer of proppant 1012 are enclosed with an enclosure 1030. An example of this step 1785 and associated details are provided above with respect to FIGS. 13 through 14B and associated description. In step 1786, test fluid 1591 flows through the layer of proppant 1512 of the modified core sample 1500. Put another way, step 1786 may involve facilitating an interaction between the subterranean core sample with the layer of proppant and a test fluid 1591, where the test fluid includes at least one component that is used during hydraulic fracturing operations. Contents of the test fluid may include, but are not limited to, biocides, friction reducers, hydrosulfide scavengers, corrosion inhibitors, iron controlling agents, surfactants, acid and acid inhibitors. An example of this step 1786 and associated details are provided above with respect to FIG. 15 and associated description. In step 1787, the layer of proppant 1512 and the exit fluid 1593 are tested and analyzed. In this step 1787, the results of the interaction of step 1786 may be measured. An example of this step 1787 and associated details are provided above with respect to FIGS. 15 and 16 and associated description. When step 1787 is complete, the process proceeds to the END step.

Example embodiments may be used to modify and/or otherwise preparing a core sample in order to test testing scale squeeze. Example embodiments may be used with core samples taken from any type (e.g., unconventional formations, conventional formations) of formation that may be subject to a fracturing operation. Example embodiments may provide a number of benefits. Such benefits may include, but are not limited to, ease of use, short commissioning time, extending the life of a producing subsea well, flexibility, configurability, and improved compliance with applicable industry standards and regulations.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method for preparing a subterranean core sample for testing scale squeeze, the method comprising:
obtaining the subterranean core sample, wherein the core sample comprises a proximal end, a distal end, and a fracture that continuously spans along its length between the proximal end and the distal end;
separating the subterranean core sample along the fracture into a first portion and a second portion;
placing a layer of proppant on a first fracture surface of the first portion that defines the fracture;
placing the second portion atop the first portion so that a second fracture surface of the second portion that defines the fracture contacts the layer of proppant, and so that the proximal end of the first portion is adjacent to the proximal end of the second portion, and so that the distal end of the first portion is adjacent to the distal end of the second portion; and
enclosing the first portion, the second portion, and the layer of proppant in between with an enclosure along the length, wherein the proximal end and the distal end are uncovered by the enclosure.

2. The method of claim 1, wherein the fracture is created by cutting the subterranean core sample along its length.

3. The method of claim 1, wherein the fracture exists in the subterranean core sample when the subterranean core sample is obtained.

4. The method of claim 1, wherein the layer of proppant is substantially uniform along the length.

5. The method of claim 1, further comprising:
facilitating an interaction between the subterranean core sample with the layer of proppant and a test fluid comprising at least one component that is used during hydraulic fracturing operations; and
measuring results of the interaction.

6. A modified core sample for testing scale squeeze, the modified core sample comprising:
a first portion of a core sample comprising a first length defined by a first proximal end and a first distal end, wherein the first portion further comprises a first fracture surface disposed along the first length;
a second portion of a core sample comprising a second length defined by a second proximal end and a second distal end, wherein the second portion further comprises a second fracture surface disposed along the second length, and wherein the second fracture surface complements the first fracture surface;
a layer of proppant placed between the first fracture surface and the second fracture surface; and
a cover that encloses the first portion, the second portion, and the layer of proppant along the first length and the second length, wherein the first proximal end, the first distal end, the second proximal end, and the second distal end are uncovered by the cover.
